(19) United States
(12) Patent Application Publication
(10) Pub. No.: US 2024/0226654 A1
(43) Pub. Date: Jul. 11, 2024

(54) Tongue Depression Auxiliary Device

(71) Applicant: Yu-Shiang Wu, Taichung City (TW)

(72) Inventors: Yu-Shiang Wu, Taichung City (TW); Chu-Wei Huang, Taichung City (TW)

(21) Appl. No.: 18/151,120

(22) Filed: Jan. 6, 2023

(51) Int. Cl. A63B 23/18

(52) U.S. Cl. CPC A63B 23/18; A61B 13/00

(57) ABSTRACT

A tongue depression auxiliary device composed of a brace and a support member, the brace is detachably sleeved in an oral cavity, the support member is connected to the brace, and the support member extends from the brace toward a lower jaw to support a tongue and push the tongue. The tongue depression auxiliary device can be worn no matter when a user is awake or sleeping and is capable of achieving an efficacy of increasing lung capacity and stimulating and activating fascia, and capable of achieving an effect of relieving stress and relaxing breathing, and improving the phenomenon of insomnia or snoring.

### BACKGROUND OF THE INVENTION

### 1. Field of Invention

The invention relates to a training device for breathing, and more particularly to a tongue depression auxiliary device capable of increasing lung capacity and reducing inhalation/ventilation frequencies.

### 2. Related Art

Generally speaking, when exercising, the muscle tissue of the human body will use adenosine triphosphate (ATP) to produce repeated contractions. As the exercise time increases, the concentration of ATP will gradually decrease, resulting in decreased muscle function and muscle fatigue. In particular, it is more likely to cause dizziness, spiritless in many people when they are doing vigorous exercise. In severe cases, it can also cause chest tightness, shortness of breath, and asthma. At present, although there are techniques to promote aerobic exercise to train lung capacity, chest expansion exercise (such as stretching) to expand thoracic cavity to train lung capacity, or adjust breathing methods to specially use abdominal breathing to train lung capacity; however, these exercises or training must be carried out consciously before they can be completed. At present, there is no technique that can perform the aforementioned vital capacity training in the conscious or unconscious state of the user.

### SUMMARY OF THE INVENTION

In view of the above mentioned, an object of the invention is to provide a tongue depression auxiliary device capable of training muscles of a human body to control the breathing behavior whether a user is conscious or not, thereby improving lung capacity and body function, and an effect of using the tongue depression auxiliary device is even more obvious during exercise, in order to solve the above-mentioned problems of the prior art.

In order to achieve the aforementioned object, the invention discloses a tongue depression auxiliary device for using in an oral cavity, the oral cavity comprises an oral cavity opening, an upper row of teeth, a lower row of teeth, a tongue, a lower jaw on the lower row of teeth close to a side of the oral cavity opening, and a uvula connected to an upper jaw away from the side of the oral cavity opening, characterized in that, the tongue depression auxiliary device is composed of a brace and a support member, wherein the brace is detachably sleeved in the oral cavity; and the support member is connected to the brace, the support member extends from the brace toward the lower jaw to support the tongue and push the tongue toward a direction of the lower jaw.

Preferably, a peripheral edge of a top side of the support member is connected with the brace, a bottom side of the support member protrudes from a bottom side of the brace and extends from the brace toward the lower jaw to push the tongue in a direction of the lower jaw and the oral cavity opening.

Preferably, the brace is detachably sleeved on the lower row of teeth.

Preferably, the brace is detachably sleeved on the upper row of teeth.

Preferably, a bottom side of the support member is in contact with the tongue, and a top side of the support member is separated from the upper jaw or the uvula with a gap.

Preferably, a top side of the support member does not protrude from a top side of the brace, and a bottom side of the support member extends from the brace toward the lower jaw and protrudes from a bottom side of the brace.

Preferably, a surface of a top side of the support member is a flow guide surface.

Preferably, a bottom side of the support member extends a length from a bottom side of the brace toward the lower jaw, and a distance by which the support member pushes the tongue toward the lower jaw corresponds to the length of the support member.

Preferably, a cross-sectional area of the support member gradually decreases from a top side toward a bottom side to form a drainage space.

Preferably, the brace comprises a denture and a socket, and the socket is located on the denture.

Preferably, the support member is made of rubber, resin or plastic material.

Preferably, the support member is made of rigidly material to force the tongue toward the lower jaw.

Preferably, the support member is made of elastic material to force the tongue toward the lower jaw.

In summary, the tongue depression auxiliary device of the invention has the following advantages:

(1) by pushing the tongue toward the lower jaw instead of just blocking the tongue from retreating, the tongue depression auxiliary device is capable of effectively expanding a cross-sectional area of a throat opening (intake area and expiratory area) to increase lung capacity, and there will be no accumulation of saliva that cannot be eliminated;

(2) by pushing the tongue toward the lower jaw instead of just preventing an airflow from vibrating soft tissues, such as the uvula, in the oral cavity the tongue depression auxiliary device can be worn in a wider range of times, whether the user is awake or sleeping, and is capable of achieving an efficacy of increasing lung capacity and stimulating fascia to activate self-healing ability, especially an effect of using the tongue depression auxiliary device is even more obvious during exercise;

(3) by increasing intake volume and expiratory volume per unit time and effectively reducing inspiratory frequency and ventilation frequency, the tongue depression auxiliary device is capable of training and improving lung capacity, strengthening physical fitness and improving muscle power output, and achieving an effect of relieving stress and relaxing breathing, and improving the phenomenon of insomnia or snoring; and

(4) by pushing the tongue toward the lower jaw, the user can be trained to change breathing habits, such as changing from oral breathing to nasal breathing, and such as changing from thoracic breathing to abdominal breathing.

In order to enable the examiner to have a further understanding and recognition of the technical features of the invention and the technical efficacies that can be achieved, preferred embodiments in conjunction with detailed explanation are provided as follows.

### BRIEF DESCRIPTION OF THE DRAWINGS

FIG. 1 is a perspective view of a tongue depression auxiliary device of the invention.

FIG. 2 is a rear view of the tongue depression auxiliary device of the invention.

FIG. 3 is a cross-sectional side view of the tongue depression auxiliary device of the invention being worn in an oral cavity.

FIG. 4 is a cross-sectional side view of a general oral cavity without wearing the tongue depression auxiliary device of the invention.

### DETAILED DESCRIPTION OF THE INVENTION

In order to understand the technical features, content and advantages of the invention and its achievable efficacies, the invention is described below in detail in conjunction with the figures, and in the form of embodiments, the figures used herein are only for a purpose of schematically supplementing the specification, and may not be true proportions and precise configurations after implementation of the invention; and therefore, relationship between the proportions and configurations of the attached figures should not be interpreted to limit the scope of the claims of the invention in actual implementation. In addition, in order to facilitate understanding, the same elements in the following embodiments are indicated by the same referenced numbers. And the size and proportions of the components shown in the drawings are for the purpose of explaining the components and their structures only and are not intending to be limiting.

Unless otherwise noted, all terms used in the whole descriptions and claims shall have their common meaning in the related field in the descriptions disclosed herein and in other special descriptions. Some terms used to describe in the present invention will be defined below or in other parts of the descriptions as an extra guidance for those skilled in the art to understand the descriptions of the present invention.

The terms such as “first”, “second”, “third” used in the descriptions are not indicating an order or sequence, and are not intending to limit the scope of the present invention. They are used only for differentiation of components or operations described by the same terms.

Moreover, the terms “comprising”, “including”, “having”, and “with” used in the descriptions are all open terms and have the meaning of “comprising but not limited to”.

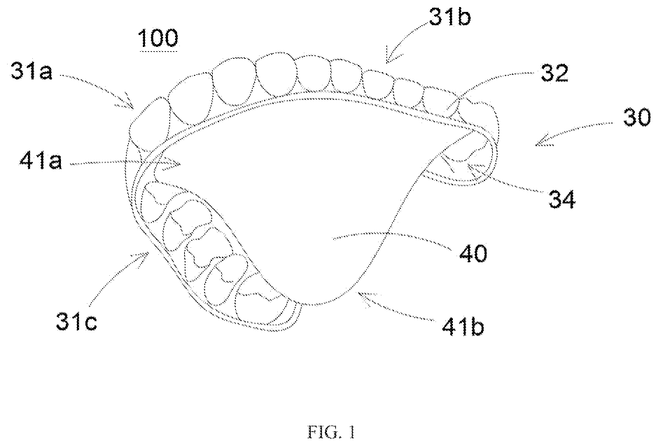

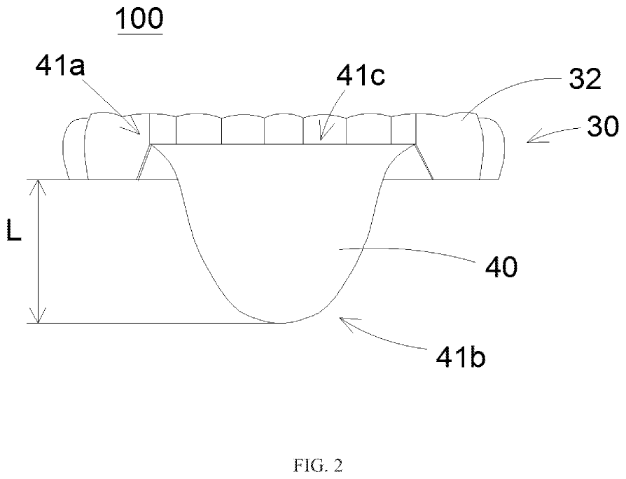

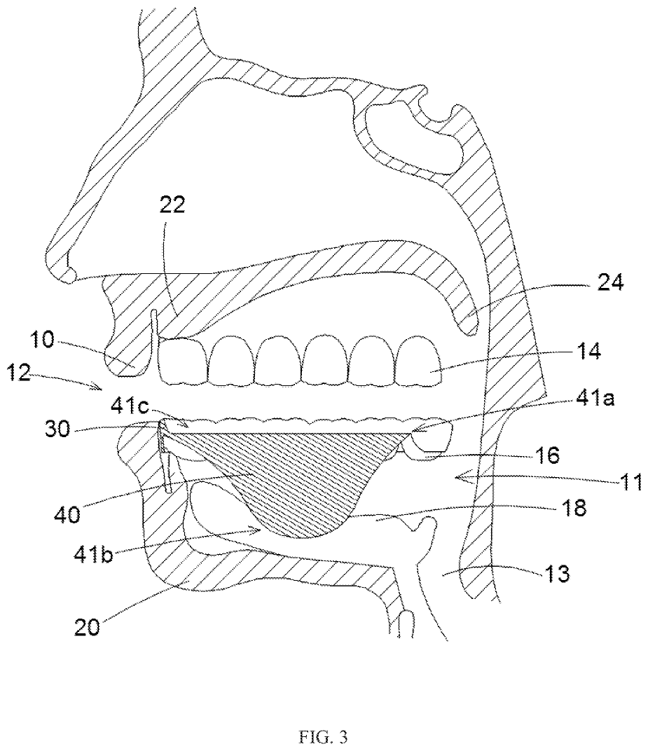

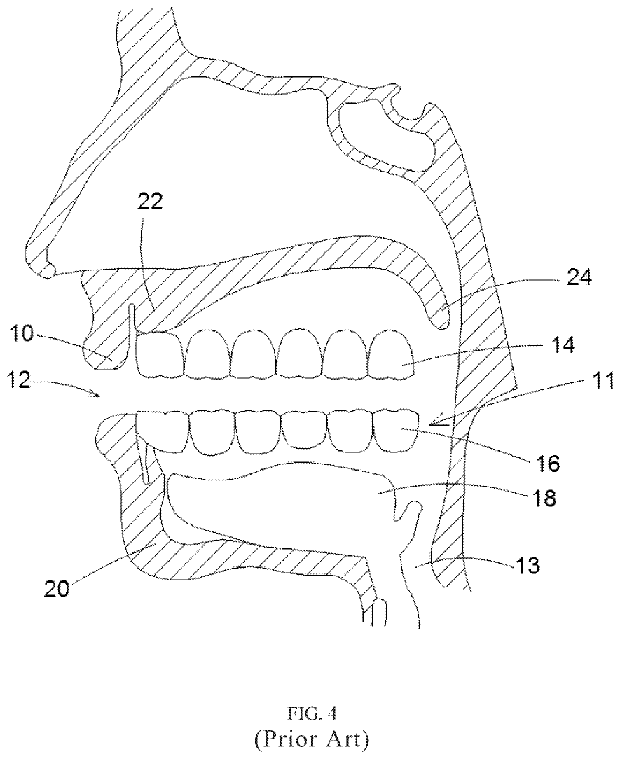

Please refer to FIG. 1 to FIG. 4. FIG. 1 is a perspective view of a tongue depression auxiliary device of the invention; FIG. 2 is a rear view of the tongue depression auxiliary device of the invention; FIG. 3 is a cross-sectional side view of the tongue depression auxiliary device of the invention being worn in an oral cavity; and FIG. 4 is a cross-sectional side view of a general oral cavity without wearing the tongue depression auxiliary device of the invention. A tongue depression auxiliary device 100 of the invention is used in an oral cavity 10, wherein the oral cavity 10 comprises an oral cavity opening 12, an upper row of teeth 14, a lower row of teeth 16, a tongue 18, a lower jaw 20 on the lower row of teeth 16 close to a side of the oral cavity opening 12, and a uvula 24 connected to an upper jaw 22 away from the side of the oral cavity opening 12.

The tongue depression auxiliary device 100 of the invention is composed of a brace 30 and a support member 40. Materials of the brace 30 and the support member 40 are, such as but not limited to rubber, resin or plastic materials. The support member 40 can be made of rigidly material to force the tongue 18 toward the lower jaw 20. Alternatively, the support member 40 can be made of elastic material to force the tongue 18 toward the lower jaw 20. Materials of the brace 30 and the support member 40 can be the same or different, and a hardness can also be the same or different. The brace 30 is detachably sleeved in the oral cavity 10, and the support member 40 is connected to the brace 30. The brace 30 is, for example, detachably sleeved on the upper row of teeth 14. In the invention, it is a preferred example that the brace 30 is sleeved on the lower row of teeth 16, but the invention is not limited thereto. The brace 30 can also be optionally and detachably sleeved on the upper row of teeth 14, or is sleeved on the upper row of teeth 14 and the lower row of teeth 16 at the same time. A peripheral edge of a top side 41a of the support member 40 is, for example, integrally connected with the brace 30, and the support member 40 can also be, for example, detachably connected with the brace 30. The support member 40 extends from the brace 30 toward the lower jaw 20 to contact with and forcibly push the tongue 18. The support member 40 can be, for example, a hollow closed bump or a solid bump (as shown in FIG. 3). A bottom side 41b of the support member 40 is, for example, in contact with a central area of the tongue 18. When the brace 30 of the invention is sleeved on the teeth, the support member 40 will forcibly push the tongue 18 toward the lower jaw 20, such as making the tongue 18 concave, or even making the tongue 18 (including tongue tip, tongue body and root of tongue) move forward in a direction of the oral cavity opening 12, that is, away from a throat 13. A throat opening 11 is a breathing passage, and the throat opening 11 is located above the throat 13 and adjacent to the uvula 24 and the tongue 18 in the oral cavity 10, so the throat opening 11 can be expanded naturally by pushing the tongue 18. In other words, a cross-sectional area of the throat opening 11 after the tongue 18 being forcibly moved is larger than a cross-sectional area of the throat opening 11 without the tongue 18 being pushed, which means that the breathing passage is smoother. One of the features of the invention is that the support member 40 not only presses the tongue 18, but the support member 40 of the invention further pushes the tongue 18 toward the lower jaw 20 to deform the tongue 18 and even to force the tongue 18 move downward/forward to expand an intake area (cross-sectional area)/an expiratory area of the throat opening 11, and also to stimulate a fascia to activate self-healing ability. The tongue depression auxiliary device 100 of the invention is capable of increasing an amount of air intake and ventilation, and also capable of reducing a frequency of inhalation and ventilation, which means that a vital capacity can be increased. When a user does exercise (especially vigorous exercise), it is not easy to feel tired, and increasing lung capacity will have a positive effect on human body functions such as the cardiovascular system, lymphatic system, and autonomic nervous system.

Another feature of the invention is that the bottom side 41b of the support member 40 protrudes from a bottom side of the brace 30, and the bottom side 41b of the support member 40 extends (protrudes) a length L from the brace 30 toward a direction of the lower jaw 20, the length L is, for example, any numerical value between about 0 cm and about 10 cm, so that the bottom side 41b of the support member 40 supports the tongue 18, and even the tongue 18 is pushed a distance toward the lower jaw 20. A distance by which the bottom side 41b of the support member 40 extends corresponds to a distance by which the tongue 18 is pushed. The greater a distance the tongue 18 is pushed, the greater an extent the throat opening 11 is expanded. The top side 41a and a surface 41c of the support member 40 are preferably not protruding from (that is, flush with or lower than) a top side of the brace 30. Yet another feature of the invention is that the support member 40 only pushes the tongue 18, does not push the upper jaw 22 or the uvula 24, and does not need to abut against the upper jaw 22 or the uvula 24, so whether the brace 30 or the support member 40 will not hinder air from entering the throat opening 11, and therefore the invention does not need additional ventilation holes. For example, when using the tongue depression auxiliary device 100, the support member 40 only contacts the tongue 18 with the bottom side 41b, and the surface 41c of the top side 41a of the support member 40 does not contact with the upper jaw 22 or the uvula 24. That is to say, the top side 41a of the support member 40 is separated from the upper jaw 22 or the uvula 24 with a gap, and the surface 41c of the top side 41a is substantially not protruding from (that is, flush with or lower than) the top side of the brace 30. The surface 41c is preferably flat, inclined or concave to form a flow guide surface, and the surface 41c is more preferably flat so as not to affect a respiratory airflow and to be capable of guiding the airflow. In addition, an outer contour of the support member 40 is downwardly convex, and can be symmetrical or asymmetrical, for example, in a conical convex shape, that is, a width (cross-sectional area) of the top side 41a of the support member 40 is larger than a width (cross-sectional area) of the bottom side 41b. Moreover, an outer contour of the support member 40 can be in any shape, as long as the bottom side 41b of the support member 40 is capable of substantially pushing the tongue 18, the support member 40 in any shape can be applied in the invention, and the surface 41c of the top side 41a of the support member 40 is preferably to have a guide surface capable of guiding airflow. Furthermore, the surface 41c of the top side 41a of the support member 40 can also be in other forms that help guide an airflow, not limited to a horizontal plane, an arcuate surface or an inclined plane, so as to avoid reducing respiratory airflow. The support member 40 can, for example, extend vertically from top to bottom in a direction from the oral cavity opening 12 toward the throat opening 11, or for example, incline from top to bottom in a direction from the oral cavity opening 12 toward the throat opening 11, that is, incline downwardly away from the oral cavity opening 12. Similarly, the support member 40 can also, for example, incline from top to bottom from an inner side toward an outer side of the oral cavity opening 12, that is, incline downwardly toward a direction close to the oral cavity opening 12. For example, a width (cross-sectional area) of the support member 40 gradually decreases from the top side 41a toward the bottom side 41b to form a drainage space. Therefore, when the support member 40 of the invention abuts against the tongue 18 with the bottom side 41b, saliva in the oral cavity 10 can automatically flow into the throat 13 for swallowing through the drainage space between the top side 41a and the bottom side 41b, so a phenomenon of accumulation of saliva that cannot be eliminated does not occur. Of course, there is no need to additionally dispose drainage holes on the support member 40, and naturally there will be no problem of dirt accumulated in the drainage holes.

The brace 30 of the tongue depression auxiliary device 100 of the invention is, for example, a U-shaped structure, which roughly matches an outer shape of the upper jaw 22 and/or the lower jaw 20 of the user. The brace 30 has a front central portion 31a, a left side portion 31b, and a right side portion 31c. The brace 30 at least comprises a denture 32 and a socket 34, wherein the socket 34 is located on the denture 32. The socket 34 can be a U-shaped groove or optionally formed with an outer shape suitable for closely fitting the user's teeth. An opening direction of the socket 34 of the bracket 32 is towards the lower row of teeth 16 (and/or can also be towards the upper row of teeth 14) to be sleeved, so that the socket 34 can be used to sleeve the brace 30 on the lower row of teeth 16 and/or the upper row of teeth 14 of the user. In the invention, the brace 30 being sleeved on the lower row of teeth 16 is used as a preferred example, so an opening direction of the socket 34 is towards the lower row of teeth 16.

The tongue depression auxiliary device 100 of the invention is capable of forcibly pushing the tongue 18 in order to open the throat opening 11 in the oral cavity 10, so an amount of air intake and oxygen intake per unit time will also increase accordingly, and therefore is capable of effectively optimizing physical fitness and increasing muscle power output. A greater extent the throat opening 11 is stretched open, the better the above-mentioned effects. Since the tongue depression auxiliary device 100 of the invention forces the tongue 18 to displace toward the lower jaw 20, it can not only exercise and activate the fascia of the tongue 18 after wearing the tongue depression auxiliary device 100 for a period of time (for example, several days), but can also train the tongue 18 to get used to a pushed state. The invention is capable of changing the user's breathing habits from oral breathing to nasal breathing. In addition, the invention is also capable of effectively training the user to get used to breathing by abdominal breathing method. In other words, the invention enables the user to completely or partially change the original thoracic breathing (TB) to abdominal breathing (AB). A gas exchange rate in the alveoli that can be carried out by abdominal breathing is higher, and inspiratory volume/ventilation volume will increase, thereby reducing a number and a frequency of breathing. Taking a normal adult's breathing rate of about 12 to 16 times per minute as an example, the tongue depression auxiliary device 100 of the invention is capable of reducing the user's breathing times per minute. It can be known that, no matter at any time, such as reading, exercising or sleeping, especially during vigorous exercise, as long as the user wears the tongue depression auxiliary device 100 in the oral cavity 10, vital capacity can be increased and inhalation/ventilation frequencies can be reduced. Naturally, it is not easy to feel tired and the user is capable of maintaining calmness and relieving stress, it will be more efficient in doing anything with better effects. Furthermore, one of the additional effects of the invention is that if the tongue depression auxiliary device 100 is worn during sleep, a cross-sectional area of the throat opening 11 will not reduce due to relaxation and retreat of the muscles of the tongue 18, so snoring can be avoided and sleep apnea can be solved. In addition, since the tongue depression auxiliary device 100 of the invention is capable of forcibly pushing the tongue 18 to open the throat opening 11 in the oral cavity 10, instead of just abutting against the tongue 18 to prevent the tongue 18 from retreating (retracting), an amount of air intake and oxygen intake per unit time will also increase accordingly, by which the user can be trained to increase lung capacity, effects are more obvious especially during exercise (especially during vigorous exercise or long-term exercise), so the tongue depression auxiliary device 100 is capable of effectively optimizing physical fitness and improving muscle power output. When the tongue 18 is pushed, because the tongue 18 as a whole is pulled, even the root of the tongue 18 or even the throat 13 will be pulled, so the corresponding fascia will be stimulated and activated, which can help the user to exert his or her own self-healing ability.

In summary, the tongue depression auxiliary device of the invention has the following advantages:

(1) by pushing the tongue toward the lower jaw instead of just blocking the tongue from retreating, the tongue depression auxiliary device is capable of effectively expanding a cross- sectional area of a throat opening (intake area and expiratory area) to increase lung capacity, and there will be no accumulation of saliva that cannot be eliminated;

(2) by pushing the tongue toward the lower jaw instead of just preventing an airflow from vibrating soft tissues, such as the uvula, in the oral cavity the tongue depression auxiliary device can be worn in a wider range of times, whether the user is awake or sleeping, and is capable of achieving an efficacy of increasing lung capacity and stimulating fascia to activate self-healing ability, especially an effect of using the tongue depression auxiliary device is even more obvious during exercise;

(3) by increasing intake volume and expiratory volume per unit time and effectively reducing inspiratory frequency and ventilation frequency, the tongue depression auxiliary device is capable of training and improving lung capacity, strengthening physical fitness and improving muscle power output, and achieving an effect of relieving stress and relaxing breathing, and improving the phenomenon of insomnia or snoring; and

(4) by pushing the tongue toward the lower jaw, the user can be trained to change breathing habits, such as changing from oral breathing to nasal breathing, and such as changing from thoracic breathing to abdominal breathing.

Note that the specification relating to the above embodiments should be construed as exemplary rather than as limitative of the present invention, with many variations and modifications being readily attainable by a person of average skill in the art without departing from the spirit or scope thereof as defined by the appended claims and their legal equivalents.

What is claimed is:

1. A tongue depression auxiliary device for using in an oral cavity, the oral cavity comprising an oral cavity opening, an upper row of teeth, a lower row of teeth, a tongue, a lower jaw on the lower row of teeth close to a side of the oral cavity opening, and a uvula connected to an upper jaw away from the side of the oral cavity opening, characterized in that the tongue depression auxiliary device is composed of a brace and a support member, wherein
the brace is detachably sleeved in the oral cavity; and
the support member is connected to the brace, the support member extends from the brace toward the lower jaw to support the tongue and push the tongue toward a direction of the lower jaw.

2. The tongue depression auxiliary device as claimed in claim 1, wherein a peripheral edge of a top side of the support member is connected with the brace, a bottom side of the support member protrudes from a bottom side of the brace and extends from the brace toward the lower jaw to push the tongue in a direction of the lower jaw and the oral cavity opening.

3. The tongue depression auxiliary device as claimed in claim 1, wherein the brace is detachably sleeved on the lower row of teeth.

4. The tongue depression auxiliary device as claimed in claim 1, wherein the brace is detachably sleeved on the upper row of teeth.

5. The tongue depression auxiliary device as claimed in claim 1, wherein a bottom side of the support member is in contact with the tongue, and a top side of the support member is separated from the upper jaw or the uvula with a gap.

6. The tongue depression auxiliary device as claimed in claim 1, wherein a top side of the support member does not protrude from a top side of the brace, and a bottom side of the support member extends from the brace toward the lower jaw and protrudes from a bottom side of the brace.

7. The tongue depression auxiliary device as claimed in claim 1, wherein a surface of a top side of the support member is a flow guide surface.

8. The tongue depression auxiliary device as claimed in claim 1, wherein a bottom side of the support member extends a length from a bottom side of the brace toward the lower jaw, and a distance by which the support member pushes the tongue toward the lower jaw corresponds to the length of the support member.

9. The tongue depression auxiliary device as claimed in claim 1, wherein a cross-sectional area of the support member gradually decreases from a top side toward a bottom side to form a drainage space.

10. The tongue depression auxiliary device as claimed in claim 1, wherein the brace comprises a denture and a socket, and the socket is located on the denture.

11. The tongue depression auxiliary device as claimed in claim 3, wherein the brace is detachably sleeved on the upper row of teeth.

12. The tongue depression auxiliary device as claimed in claim 5, wherein the top side of the support member does not protrude from a top side of the brace, and the bottom side of the support member extends from the brace toward the lower jaw and protrudes from a bottom side of the brace.

13. The tongue depression auxiliary device as claimed in claim 5, wherein a surface of the top side of the support member is a flow guide surface.

14. The tongue depression auxiliary device as claimed in claim 1, wherein the support member is made of rubber, resin or plastic material.

15. The tongue depression auxiliary device as claimed in claim 1, wherein the support member is made of rigidly material to force the tongue toward the lower jaw.

16. The tongue depression auxiliary device as claimed in claim 1, wherein the support member is made of elastic material to force the tongue toward the lower jaw.
